(19) United States
(12) Patent Application Publication
(10) Pub. No.: US 2017/0042822 A1
(43) Pub. Date: Feb. 16, 2017

(54) NON-STICKY NANOFIBROUS MEMBRANE HAVING CORE AND SHELL AND PROCESS OF PRODUCING SAME

(71) Applicants: Chih-Hao Chen, Taoyuan City (TW); Jyh-Ping Chen, Taoyuan City (TW); Shih-Hsien Chen, Kaohsiung City (TW); Chien-Tzung Chen, Taoyuan City (TW); Victor Bong-Hang Shyu, Taoyuan City (TW)

(72) Inventors: Chih-Hao Chen, Taoyuan City (TW); Jyh-Ping Chen, Taoyuan City (TW); Shih-Hsien Chen, Kaohsiung City (TW); Chien-Tzung Chen, Taoyuan City (TW); Victor Bong-Hang Shyu, Taoyuan City (TW)

(21) Appl. No.: 14/822,362

(22) Filed: Aug. 10, 2015

(51) Int. Cl. A61K 9/48

(52) U.S. Cl. CPC A61K 9/4816; A61K 9/4833

(57) ABSTRACT

A process of producing a non-sticky nanofibrous membrane includes pouring dimethylformamide (DMF) and dichloromethane into a first flask; pouring about 1 to about 40 wt % of polycaprolactone (PCL) into the first flask to prepare a PCL solution therein; keeping the first flask in a predetermined temperature wherein volumetric ratio of DMF and dichloromethane is 1:9 to 9:1; pouring hyaluronic acid (HA) into a second flask to prepare an HA solution having 1.75 wt % of HA; and activating an electrospinning technique to process the PCL solution in the first flask and the HA solution in the second flask, thereby producing an NFM having a core and a shell.

### BACKGROUND OF THE INVENTION

1. Field of the Invention

The invention relates to nanofibrous membrane (NFM) and more particularly to a process of producing the NFM by means of an electrospinning technique, the NFM having a polycaprolactone (PCL) shell and a hyaluronic acid (HA) core, the NFM being capable of inhibiting the growth of microorganisms, causing no sticky tissues growth between the tendon being healed and the surrounding tissue, and preventing the tendon from being infected after surgery.

2. Description of Related Art

Sticky tissues growth after surgery has bothered surgeons for a long time. The sticky tissues represent there is inflammation in the healed portion of the body. The sticky tissues can attract the fibrous cells surrounding tissues to heal the injured portion. However, it may grow excessive fibrous structures which stick to the surrounding tissues. It is known that more than 93% of patients may have sticky tissues after subjecting to abdominal cavity surgery. To the worse, the abdominal cavity may suffer chronic pain, sterilization, etc. Sticky tissues may grow between the tendon and the surrounding tissues after subjecting the tendon to a surgery. The sticky tissues can hinder the toggle movement of the joint.

Using an electrospinning technique to process a biodegradable biomedical material for preventing the growth of sticky tissues is rare. Currently, there is disclosure of using an electrospinning technique to process co-polymers including poly(lactic-co-glycolic acid) (PLGA), polycaprolactone (PCL), polyethylene glycol (PEG) and polylactic acid (PLA), and mixed electrospun membranes of chitosan and algin to produce non-sticky tissues for healing wounds after abdominal cavity surgery. Currently, there are researches about using eletrospinning technique to process a biodegradable biomedical material such as PCL fibers containing HA particles, PEG, and PLA which are mixed to form ibuprofen, and PLA mixed with nano porous silicon particles containing ibuprofen in order to solve the problem of sticky tissues growth around the tendon after surgery.

In view of above literature, it is found that simple high molecular material or single medicine release is used to decrease the sticky tissues growth. However, it cannot solve the problems including infection, inflammation, and resistance to foreign objects associated with the surgery. Typically, infection peak occurs within three days after surgery. Currently, injecting antibiotics into the whole body is the most effective method. However, it can cause side effects on the whole body.

The invention discussed below aims at solving the above problems of infection within three days after surgery and side effects by providing a nanofibrous membrane being capable of decreasing the side effects and causing no sticky tissues growth between the tendon being healed and the surrounding tissue.

### SUMMARY OF THE INVENTION

It is therefore one object of the invention to provide a process of producing a nanofibrous membrane (NFM) comprising the steps of pouring dimethylformamide (DMF) and dichloromethane into a first flask; pouring about 1 to about 40 wt % of polycaprolactone (PCL) into the first flask to prepare a PCL solution therein; keeping the first flask in a predetermined temperature wherein volumetric ratio of DMF and dichloromethane is 1:9 to 9:1; pouring hyaluronic acid (HA) into a second flask to prepare an HA solution having 1.75 wt % of HA; and activating an electrospinning technique to process the PCL solution in the first flask and the HA solution in the second flask, thereby producing an NFM having a core and a shell.

It is another object of the invention to provide a nanofibrous membrane (NFM) comprising a polycaprolactone (PCL) shell; and a hyaluronic acid (HA) core surrounded by the PCL shell.

The above and other objects, features and advantages of the invention will become apparent from the following detailed description taken with the accompanying drawings.

### BRIEF DESCRIPTION OF THE DRAWINGS

FIG. 1 is a perspective view in part section of a nanofibrous membrane (NFM) according to the invention;

FIG. 1A is a flow graph illustrating a process of producing the NFM according to the invention;

FIG. 1B is another flow graph illustrating a process of producing the NFM according to the invention;

FIG. 1C is a table showing different membranes and fiber diameters and pore diameters thereof;

FIG. 2 shows six images in which part (a) is a SEM image of PCL NFM, part (b) is a SEM image of HA/PCL NFM, part (c) is a SEM image of HA/PCL+Ag NFM, part (d) is a SEM image of water contact angle of HA/PCL NFM, part (e) is a SEM image of water contact angle of HA/PCL+Ag NFM, and part (f) is an enlarged image of part (e);

FIG. 3 shows two charts in which part (a) plots time versus cumulative release for HA/PCL curve and HA/PCL+Ag curve; and part (b) plots time versus cumulative release for HA/PCL+Ag curve;

FIG. 4 is a SEM image showing growth of microorganisms of E. coli inhibited by PCL, HA/PCL, and HA/PCL+Ag, and growth of microorganisms of S. aureus inhibited by PCL, HA/PCL, and HA/PCL+Ag;

FIG. 5 shows four SEM images of the tendon being healed by PCL NFM, HA/PCL NFM, and HA/PCL+Ag NFM, and a control, the SEM images being taken three weeks after surgery and the arrow pointing to the sticky tissues;

FIG. 6 shows four SEM images of a control, the tendon (T), the membrane (M), and the suture site (S), the tendon being healed by PCL NFM, HA/PCL NFM, the SEM images being taken three weeks after surgery, the arrow pointing to the sticky tissues;

FIG. 7 shows two bar charts for evaluating the non-sticky property of the NFM in terms of bending degree of the toe joint three weeks after the surgery in which part (a) shows degree versus control, PCL, HA/PCL, and HA/PCL+Ag for the joint of the first toe, * shows a great difference with the control, # shows a great diffence with PCL

NFM, and δ shows a great difference with HA/PCL NFM; and part (b) shows degree versus control, PCL, HA/PCL, and HA/PCL+Ag for the joint of the second toe, * shows a great difference with the control, # shows a great difference with PCL NFM, and δ shows a great difference with HA/PCL NFM; and

FIG. 7A shows three bar charts for evaluating the non-sticky property of the NFM in terms of sliding distance of the tendon, pull-out force, and mechanical strength of the tendon three weeks after the surgery in which part (c) shows degree versus control, PCL, HA/PCL, and HA/PCL+Ag for the sliding distance of the tendon, * shows a great difference with the control, # shows a great difference with PCL NFM, and δ shows a great difference with HA/PCL NFM; part (d) shows degree versus control, PCL, HA/PCL, and HA/PCL+Ag for the pull-out force, * shows a great difference with the control, # shows a great difference with PCL NFM, and δ shows a great difference with HA/PCL NFM; and part (e) shows degree versus control, PCL, HA/PCL, and HA/PCL+Ag for the mechanical strength of the tendon, * shows a great difference with the control, # shows a great difference with PCL NFM, and δ shows a great difference with HA/PCL NFM.

### DETAILED DESCRIPTION OF THE INVENTION

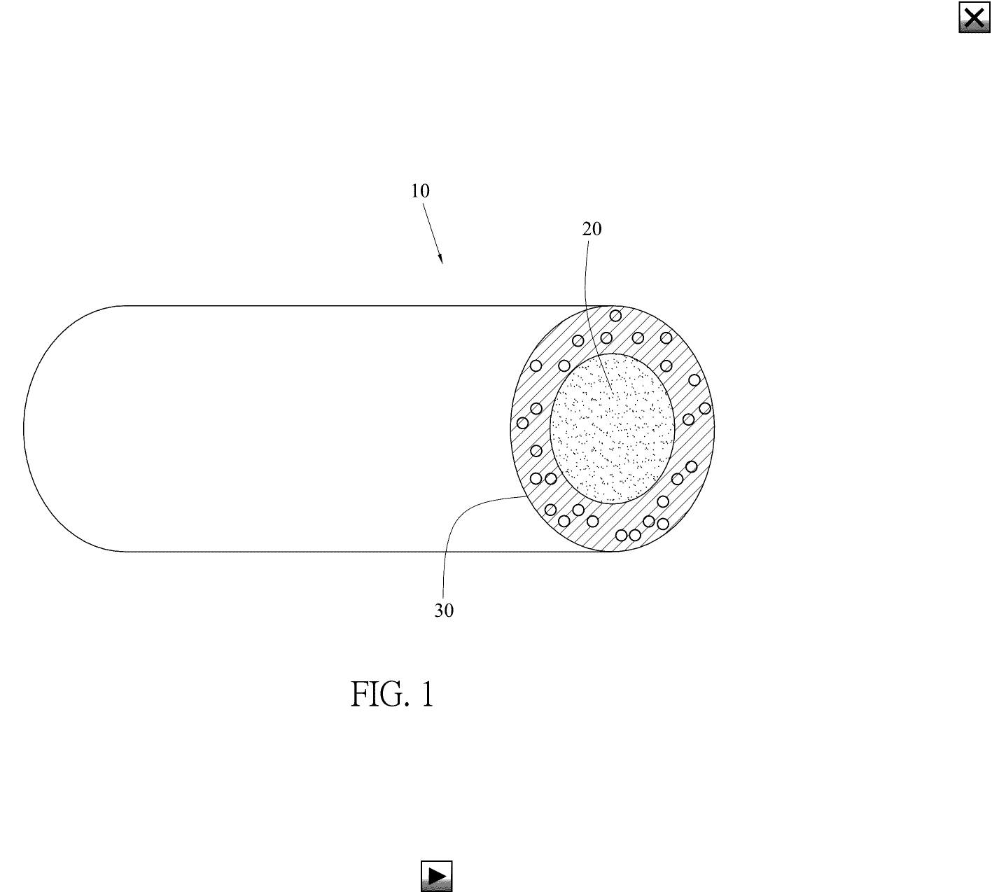

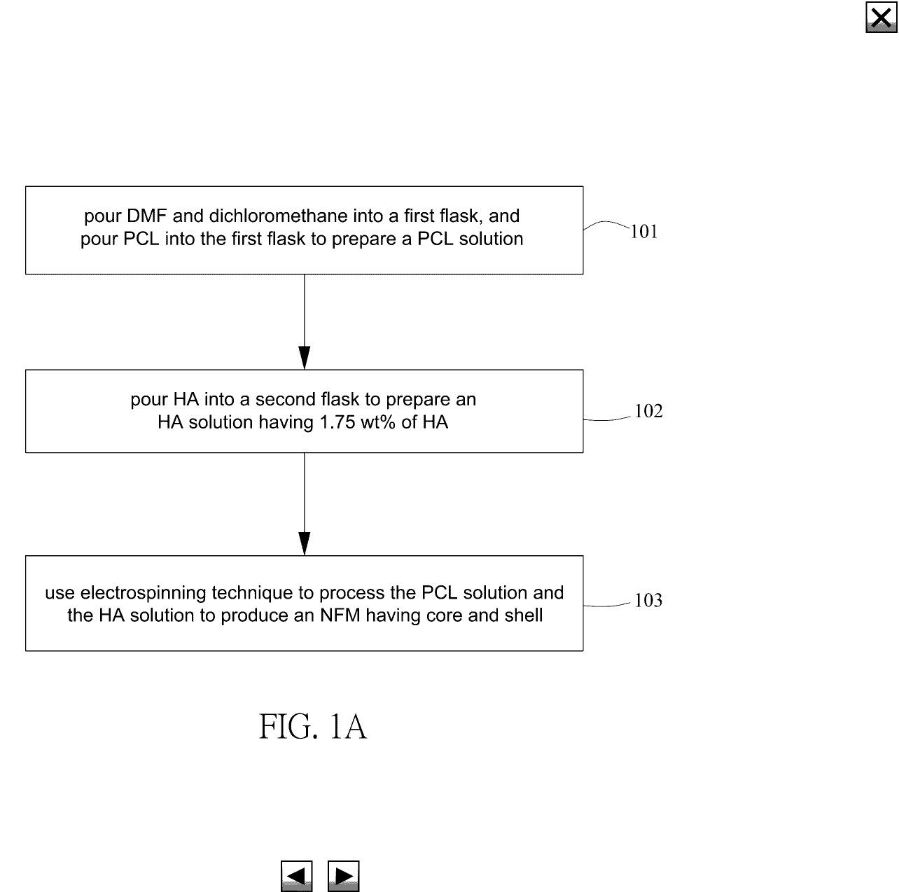

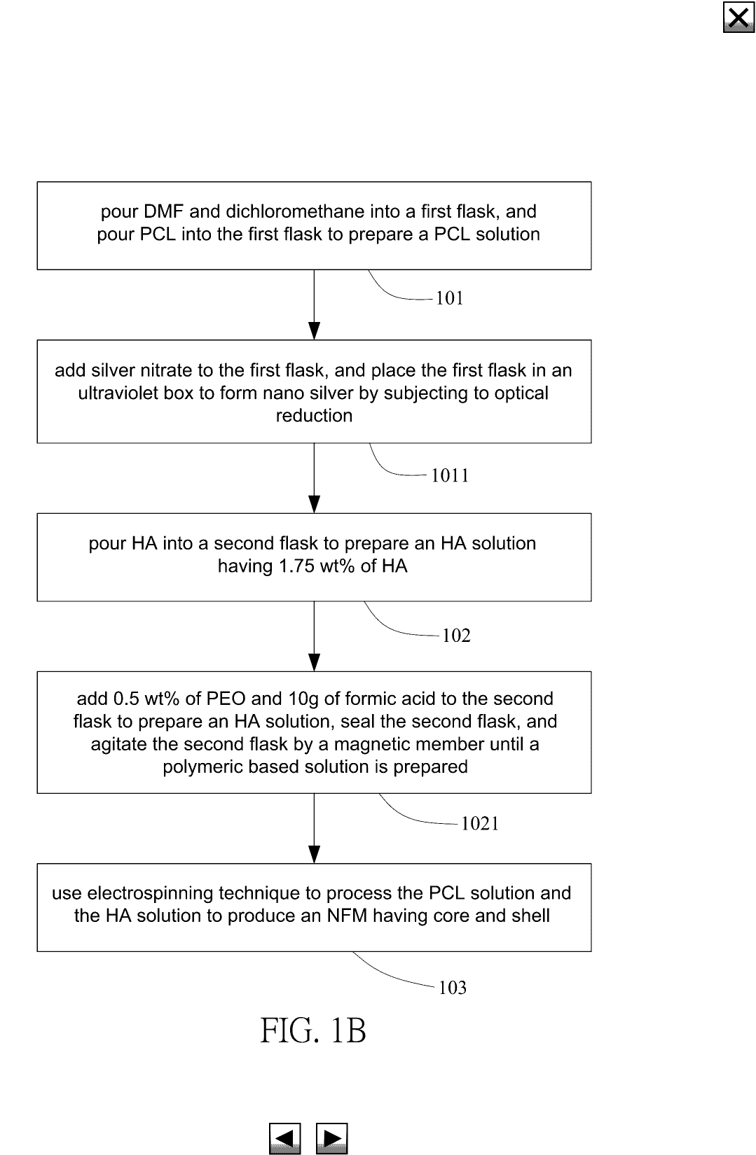

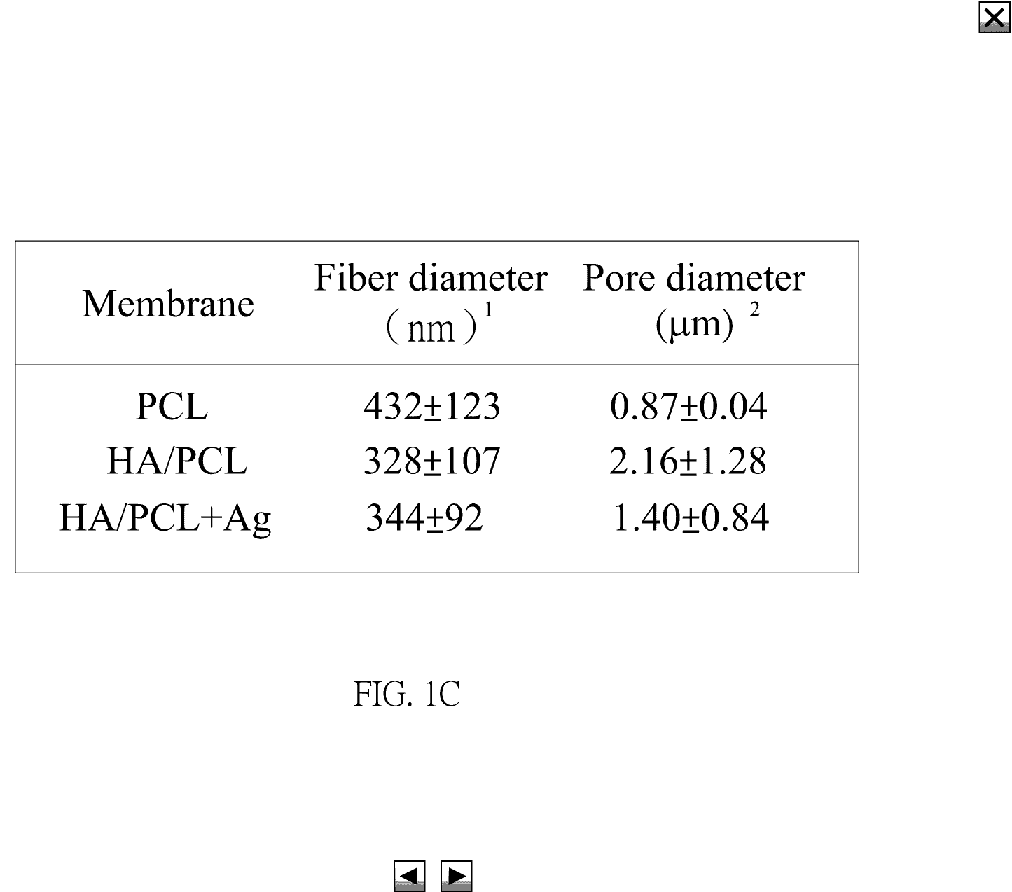

Referring to FIGS. 1 to 7A, a process of producing non-sticky NFM in accordance with the invention is illustrated below. The process comprises the following steps:

Step 101, a solution for producing shell of NFM 10 is prepared. In detail, dimethylformamide (DMF) and dichloromethane are poured into a first flask. Next, pour about 1 to about 40 wt % of polycaprolactone (PCL) into the first flask for solving. Next, a PCL solution is prepared in the first flask and placed in a room temperature environment after they are completely solved. Volumetric ratio of DMF and Dichloromethane is 1:9 to 9:1.

Step 102, a solution for producing core of the NFM 10 is prepared. In detail, hyaluronic acid (HA) is poured into a second flask to prepare an HA solution having 1.75 wt % of HA.

Step 103, an electrospinning technique is used to produce an NFM 10 having both core 20 and shell 30 by processing the PCL solution in the first flask and the HA solution in the second flask.

In sub-step 1011 of step 101, about 0.01 to about 10 wt % of silver nitrate is added to the first flask in step 101. Next, the first flask is placed in an ultraviolet box to be radiated for 3 hours to form nano silver by subjecting to optical reduction. The conditions of the ultraviolet box are 254 nm, 115V, 60 Hz, 0.7A and 0.1 J/cm².

In detail of step 101, volumetric ratio of DMF and Dichloromethane is 2:8. 0.8 g of PCL is added to the first flask. The solution has 8 wt % of PCL and 0.5 wt % of silver nitrate.

In sub-step 1021 of step 102, PCL is about 1.75 wt % and about 0.5 wt % of polyethylene oxide (PEO) and about lOg of formic acid are added to the second flask to prepare an HA solution. Next, the second flask is sealed and agitated by a magnetic member until a polymeric based solution is prepared.

In step 103, a coaxial double needle is pierced into the first and second flasks respectively. Next, solution in each of the first and second flasks is drawn into a syringe. An adapter is provided on the syringe to connect to an end of Teflon pipe. An outer needle of the double needle at the other end of the Teflon pipe is provided with a stainless needle having a bore of 16-25 and an inner needle of the double needle at the other end of the Teflon pipe is provided with a stainless needle having a bore of 18-27. The inner and outer needles are secured to two syringe pumps respectively with different flow rates being set in the syringe pumps. Flow rate of the solution containing core is about 0.1-20 mL/hr and flow rate of the solution containing shell is about 0.3-30 mL/hr. The coaxial double needle is electrically connected to positive terminal of a high voltage output. The inner and outer needles are concentric. Liquid flows out of the inner and outer needles respectively. Collector for collecting nanoscale material is grounded. Finally, the syringe pump is adjusted so that flow rates of liquid through the inner and outer needles are equal at a value of about 1.0 mL/hr. Output voltage is 20 kV. Distance between the stainless needle and the collector is about 15 cm. As a result, NFM having core and shell (not shown) is produced.

Preferably, the sharp point of the inner needle has a bore of 23, the sharp point of the outer needle has a bore of 18, and the collector is a diaphragm of aluminum.

Preferably, the PCL has a molecular weight of 1,000-1,000,000 Da.

Preferably, the NFM has a thickness of 1-5,000 nm, diameter of 10-300,000 nm, bore of 30-50,000 nm, and pore for permeability of 0.01-300,000 nm.

The non-sticky NFM 10 of the invention has a core 20 and a shell 30 within the core 20 in which the core 20 is HA and the shell 30 is PCL.

Preferably, silver nitrate is added to the shell 30 of PCL.

Preferably, PEO is added to the core 20 of PA.

Preferably, the NFM 10 has a diameter of 10-300,000 nm.

Preferably, the NFM 10 has a pore diameter of 50-50,000 nm.

Preferably, the NFM 10 has a porosity of 30-99%.

The PCL component of the NFM 10 has the effect of slowly releasing HA. Nano silver quickly releases PCL, and decreases undesired side effects. PEO added to the core 20 has the advantages of increasing reliability of the process. As a result, the NFM 10 having a non-sticky property for tendon and being capable of inhibiting the growth of microorganisms is produced (see FIG. 1).

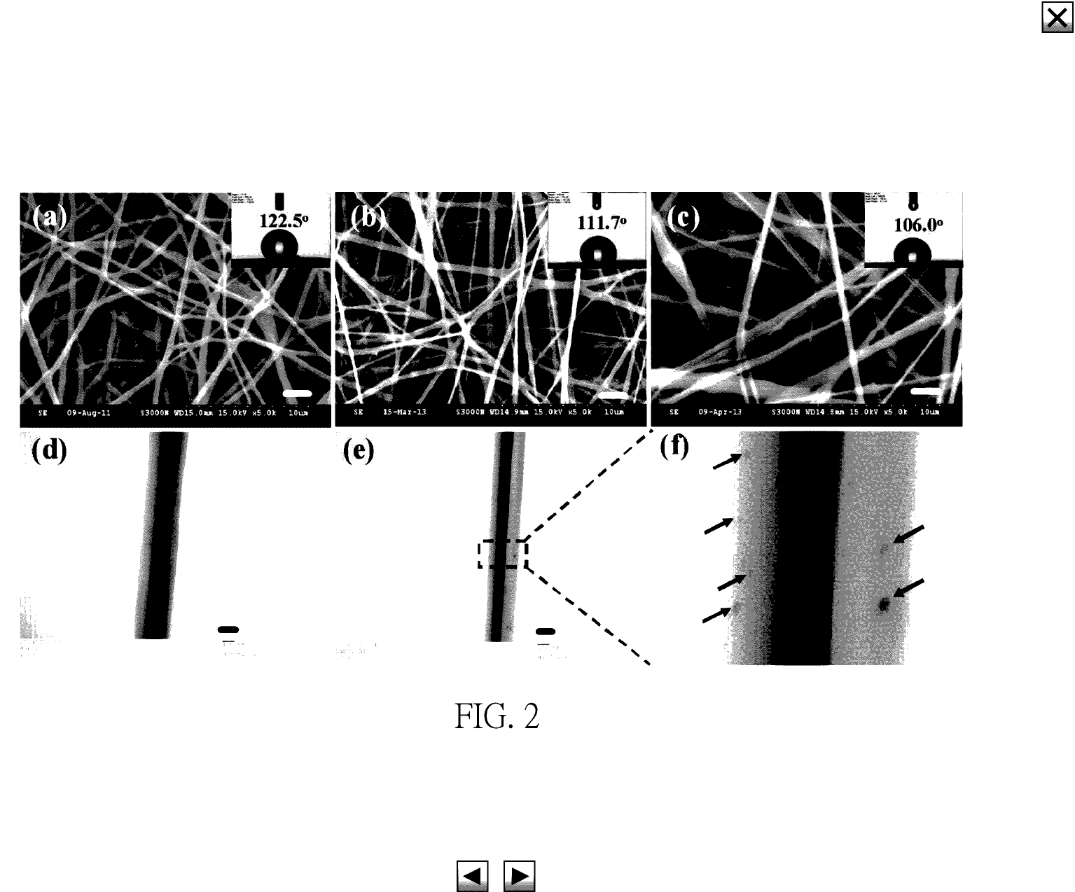

Core and shell of the NFM having HA/PCL and Ag each has an average diameter of 344±92 nm (see parts (d) to (f) of FIG. 2). Scanning electron microscope (SEM) is used to produce image of the NFM produced by the coaxial double needle of electrospinning in order to observe the fibrous structure of the NFM. As shown in parts (d) and (e) of FIG. 2, center of the fiber is black and two sides thereof are gray. It is found that fibrous structures of core and shell are shown in each of parts (d) and (e) of FIG. 2.

As shown in part (f) of FIG. 2, there are some speckles on the shell and the speckles are converted into nano silver particles after optical reduction. As shown in FIG. 1C, the NFM 10 has a pore diameter of 0.87 to 2.16 nm by means of SEM. Sizes of the fiber cells are 8-10 nm. It is confirmed that the three types of NFM 10 can effectively prohibit the penetration of the fiber cells. As shown in part (a) of FIG. 2, water contact angle of PCL NFM is 122.5 degrees of water flow. As shown in parts (b) and (c) of FIG. 2, water contact angles of HA/PCL and HA/PCL+Ag are decreased to 111.7 and 106.0 degrees of hydrophobicity respectively. This is because HA in the core is hydrophilic.

HA and Ag release confirmed by experiment:

Prepared nanofiber is cut into disc-shaped membranes having a diameter of 1.6 cm. Next, the membranes are poured into a flask of 20 mL. Next, phosphate of 3 mL having a pH of 7.4 is poured into the flask. Next, the flask is heated to 37° C. and the solution in the flask is regularly observed. Next, enzyme-linked immunosorbent assay (ELISA) is conducted to determine concentration of HA in the solution of the flask. Further, sensor coupled plasma emission spectrometer is used to determine concentration of silver ions.

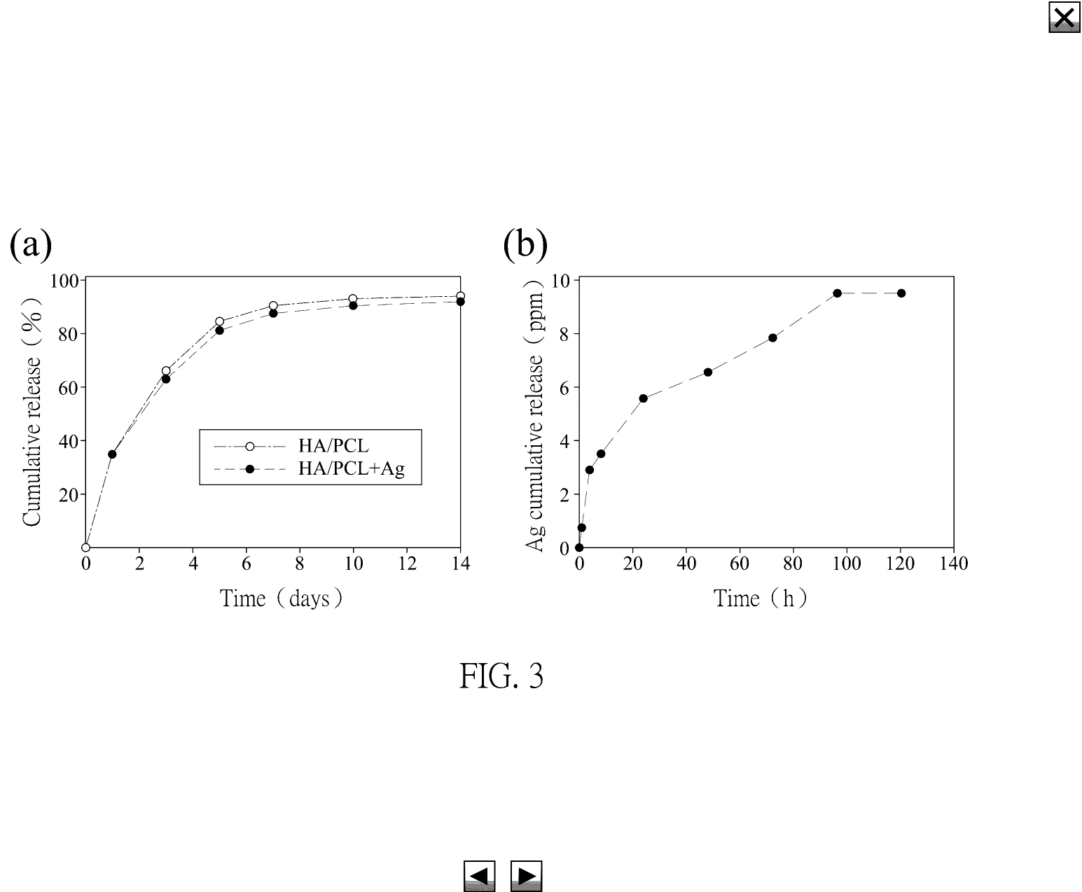

As shown in part (a) of FIG. 3, release rate of HA in each of HA/PCL and HA/PCL+Ag NFM is large and stable in the first seven days. 90% of HA of the solution is released after 10 days. HA is highly soluble in water. It is thus proved that PCL in the shell 30 can effectively decrease the release rate of HA. Further, HA release rates of HA/PCL and HA/PCL+Ag NFM are about the same because core-shell ratios of HA/PCL and HA/PCL+Ag NFM are about the same. Release of HA can smooth tissues between tendon and fiber membranes. As a result, the NFM is made non-sticky. As shown in part (b) of FIG. 3, silver ions are released continuously from HA/PCL+Ag NFM in first 96 hours and silver ions are not released thereafter. This is similar to continuously injecting antibiotics for 72 hours. Thus, these two properties of the NFM can be used as medicine for inhibiting the growth of microorganisms and as being non-sticky to other tissues.

### Test to Prove Inhibition of the Growth of Microorganisms by the NFM 10:

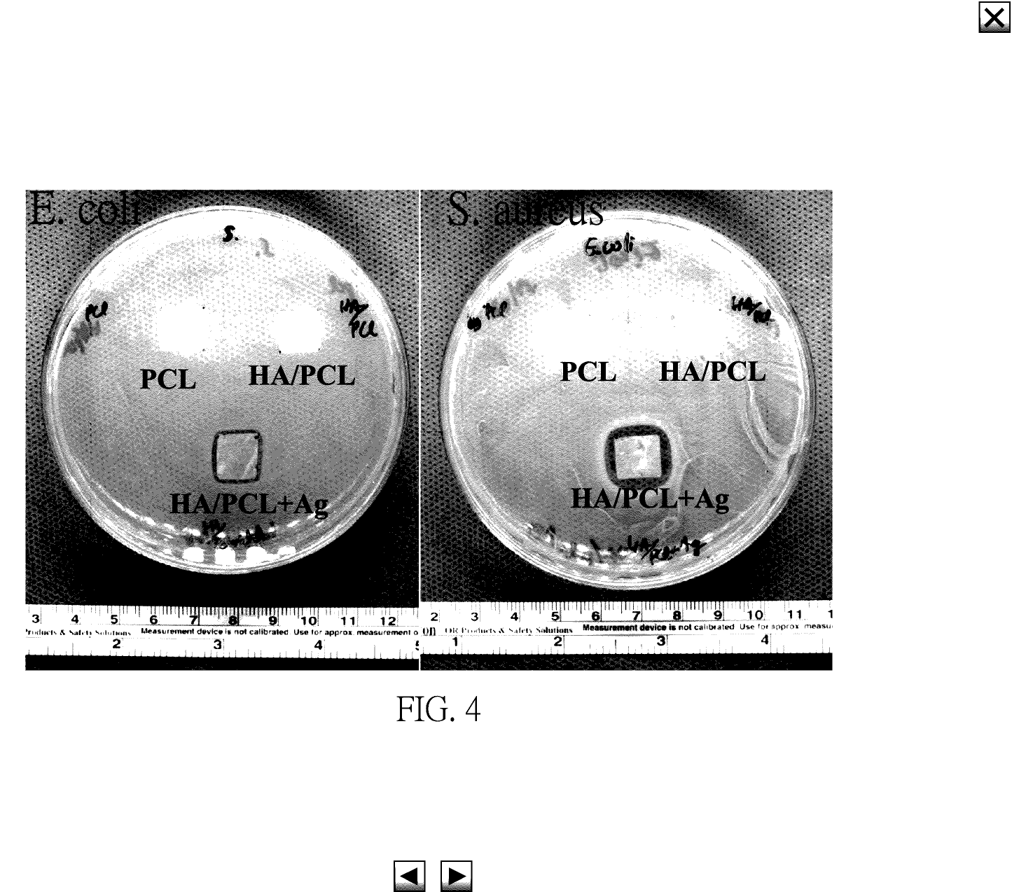

For determining whether the released silver ions have the property of inhibiting the growth of microorganisms, Gram-positive bacteria and Gram-negative bacteria are used as test targets. Techerichia coli (E. coli) is taken as Gram-negative bacteria and Staphylococcus aureus (S. aureus) is taken as Gram-positive bacteria. As shown in FIG. 4, HA/PCL+Ag NFM can inhibit the growth of microorganisms in E. coli and S. aureus in which E. coli has an area of 2.21±0.14 cm² and S. aureus has an area of 1.66±0.02 cm². To the contrast, PCL and HA/PCL NFM do not inhibit the growth of microorganisms in E. coli and S. aureus. This can prove that silver ions released by HA/PCL+Ag NFM can inhibit of the growth of microorganisms.

### Test to Prove Non-Sticky Property of the NFM 10:

We take the deep tendon around ankle of a foot of a rabbit having weight of 2-3 kg as experiment target. First, thin tendon around ankle is removed. Next, the deep tendon around ankle is cut. The deep tendon is sutured by means of improved Kessler which simulates a surgical operation. Next, the NFM 10 is wrapped around the sutured tendon to prevent sticky liquid from being grown between the sutured tendon and the surrounding tissues. The tendon not wrapped around by the NFM 10 is taken as control. The experiment takes three weeks. Thereafter, distal interphalangeal joint angle, proximal interphalangeal joint angle, and pull-out force are taken as quantitative criteria for determining stickiness. Next, images and dyed sliced tissues are taken as qualitative criteria for determining stickiness. Prior to the experiment, ethylene oxide is used to inhibit the growth of microorganisms in the fiber membrane which is later kept in a sterilized bag. Next, the tendon around ankle is removed and tested by a tension tester for determining breakage strength and tensile strength. The healed tendon around ankle after the wrapping is next compared with an injured tendon around ankle recovered naturally for effectiveness determination.

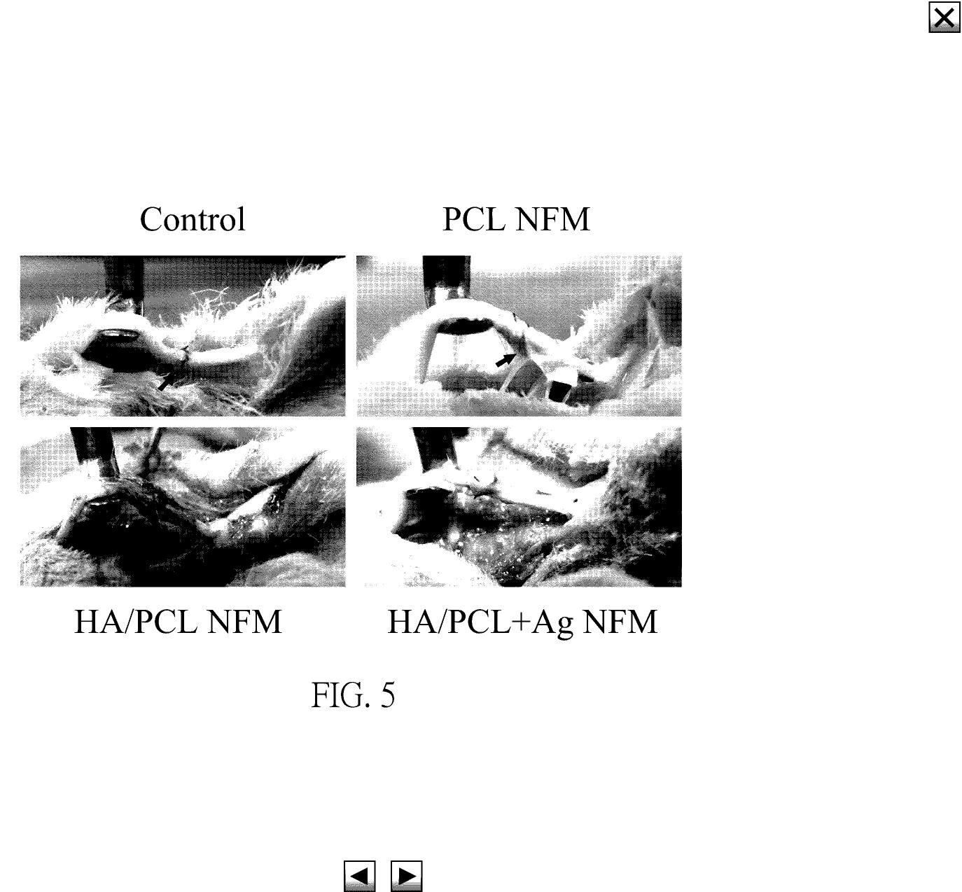

The non-stickiness of the tendon around ankle is observed through images after three weeks of the surgical operation. In FIG. 5, four images are shown. It is found that no sticky tissues are grown between the tendon and the surrounding tissues in which the tendon is wrapped around by HA/PCL+Ag NFM. This means that the tendon and the surrounding tissues can be separated naturally. In the control, it is found that sticky tissues are grown between the tendon and the surrounding tissues in which the tendon is not wrapped around by HA/PCL+Ag NFM. The tendon is completely wrapped around by the sticky tissues grown from the fiber membranes. Thus, a great force is required to separate the tendon and the surrounding tissues. Regarding the set wrapped around by PCL, there are some loose links interconnecting the tendon and the fiber membranes. Thus, a less strong force is still required to separate the tendon from the surrounding tissues. Regarding the set wrapped around by HA/PCL, there are few loose links interconnecting the tendon and the surrounding tissues. Thus, a much less force is required to separate the tendon from the surrounding tissues.

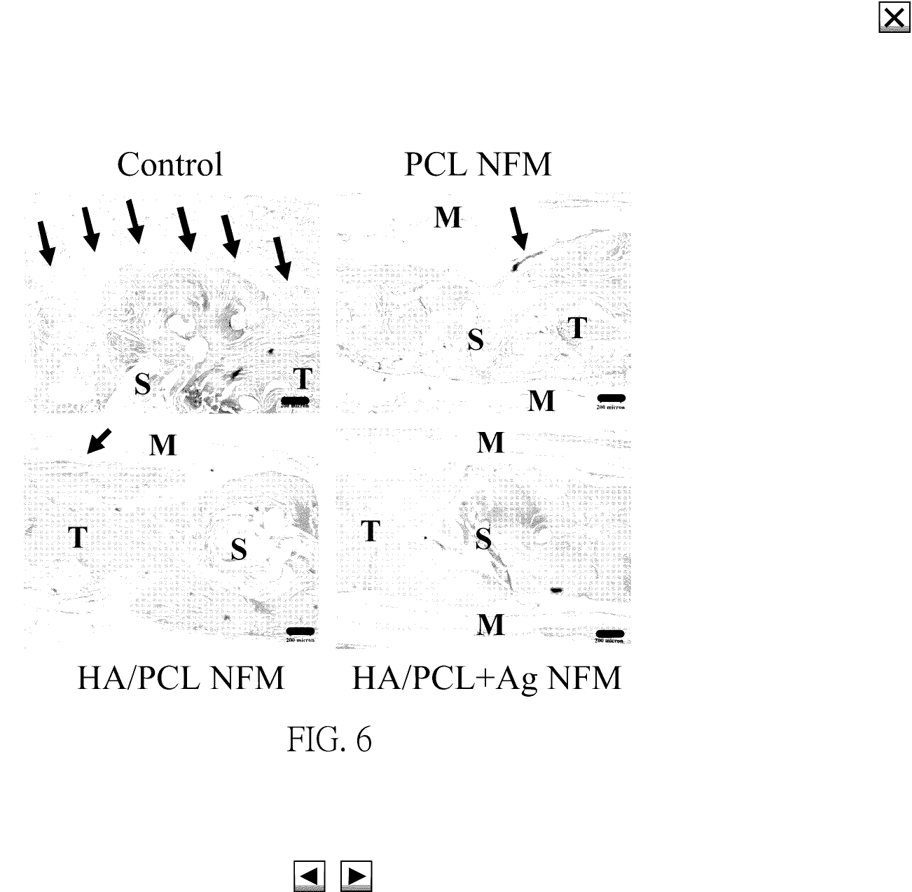

In FIG. 6, representative dyed sliced tissues of the tendon healed by HA/PCL+Ag NFM are shown. In the tendon healed by HA/PCL+Ag NFM, there are no sticky tissues are grown between the tendon and the surrounding tissues. It can be found that the tendon healed by HA/PCL+Ag NFM has an excellent non-sticky property by viewing the SEM images. Thus, it is possible of preventing sticky tissues from growing between the tendon and the surrounding tissues (see FIGS. 5 and 6). In FIG. 6, representative dyed sliced tissues of tendon sets are shown. It is found that in control there are many sticky tissues are grown between the tendon and the surrounding tissues. Budding structures having tiny blood vessels can be found in the sticky tendon. The sticky fiber membranes invade epitenon and tendon's thin layers degrade collagen. In the PCL NFM set, it is found that loose fiber structures link the healed tendon. In the HA/PCL NFM set, it is also found that loose fiber structures link the healed tendon.

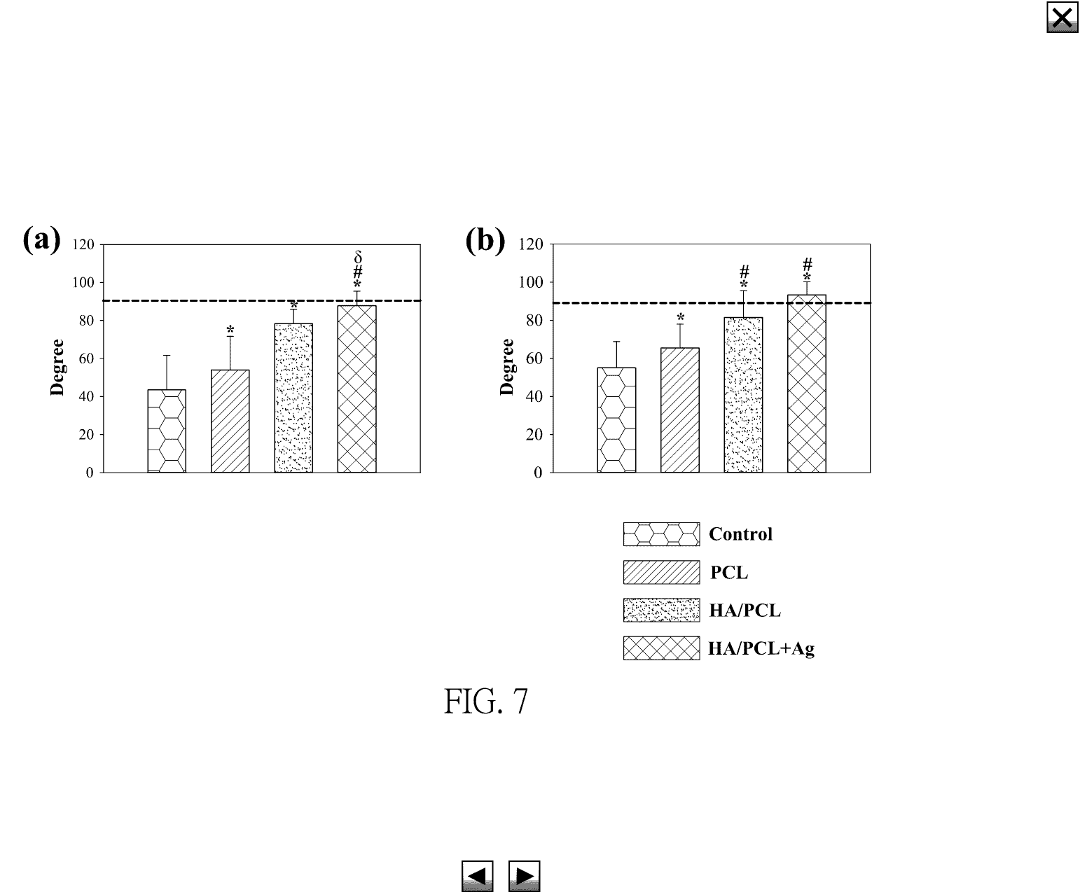

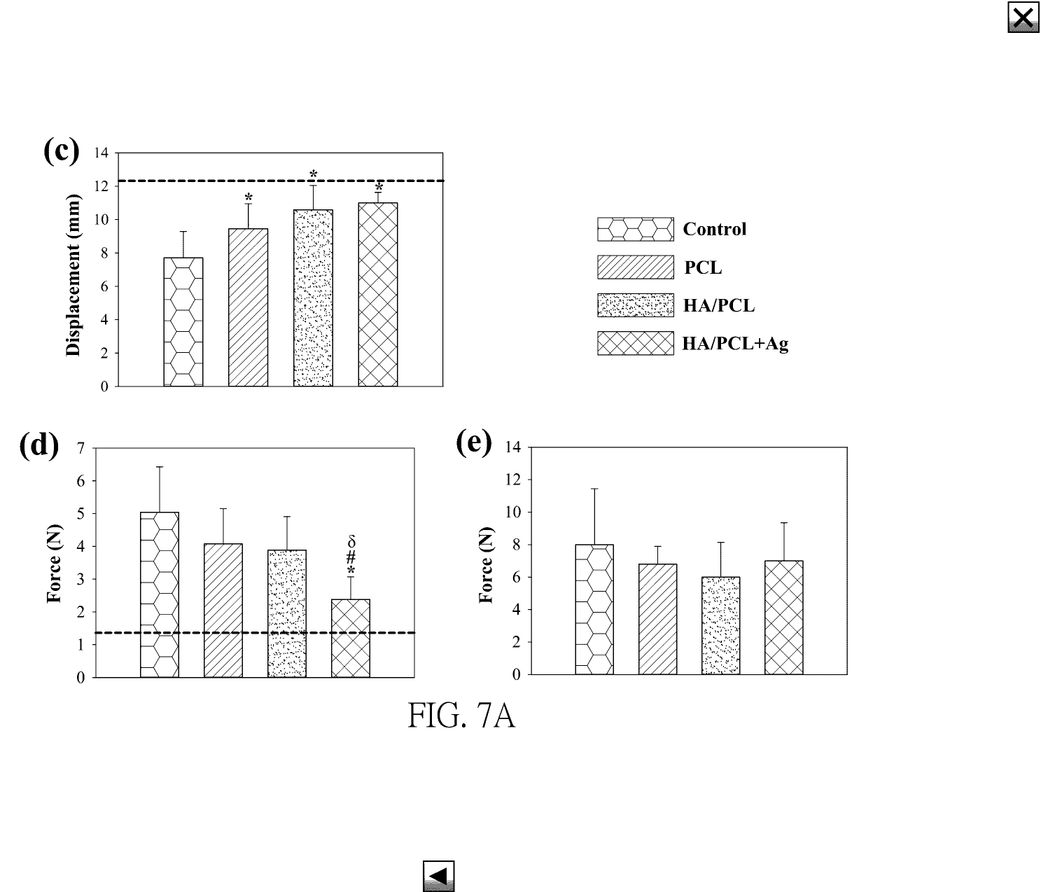

For assessing the non-sticky property of different membranes in the tissues, we test joint bending angle, tendon sliding distance, and biological mechanics of the rabbit. First, FDP tendon wrapped by PCL, HA/PCL, or HA/PCL+Ag NFM of a rabbit is compared with FDP tendon not wrapped by PCL, HA/PCL, or HA/PCL+Ag NFM of another rabbit as control. DIP and PIP joint toggle range are mainly used to determine whether there is a restriction on the joint. In comparison with the control, DIP angle (see part (a) of FIG. 7), PIP angle (see part (b) of FIG. 7), and tendon sliding distance (see part (c) of FIG. 7) of the experiment set are greatly increased. But only the HA/PCL+Ag NFM set can recover to DIP angle, PIP angle, and tendon sliding distance of normal FDP tendon (see FIGS. 7 and 7A). In a typical healing process, the surrounding tissues may stick to the tendon, thereby adversely affecting the sliding distance and the bending angle. We can determine the optimum set for preventing sticky tissues from growing on the tendon by testing different membrane wrappings in terms of sliding distance and bending angle. Regarding the effect of preventing sticky tissues from growing on the tendon, HA/PCL+Ag NFM>HA/PCL>PCL after comparing DIP angles, PIP angles, and tendon sliding distances of the different sets. Regarding DIP bending angle, the HA/PCL+Ag NFM set is greatly different from other sets. Regarding PIP being angle, the HA/PCL+Ag NFM set is also greatly different from the PCL set and the control. Regarding the tendon sliding distance, all sets wrapped by NFM are greatly different from the control.

In part (d) of FIG. 7A, force (N) versus control, PCL, HA/PCL, and HA/PCL+Ag is shown. This test method is particularly related to the sticky extent of the tendon. As expected, the control, i.e., the tendon not healed by the invention, the greatest force is required to separate the tendon from the surrounding tissues. Regarding other three sets, the least force is required to separate the tendon and the surrounding tissues when HA/PCL+Ag NFM is applied; and median force is required to separate the tendon and the surrounding tissues when HA/PCL or PCL NFM is applied. In general, some improvements are made by HA/PCL and PCL NFM sets, and the greatest improvement is made by HA/PCL+Ag NFM set. Only HA/PCL+Ag NFM can exert a force about the same as that required for normal FDP tendon. There is no difference among four sets in terms of mechanical strength test of the healed tendon after three weeks of the surgical operation (see part (e) of FIG. 7A). Breakage strength means effectiveness of the healed tendon. This means the progress of the healed tendon is the same for all four sets with tendon wrapping being considered or not. This means the progress of the healed tendon is not relevant to the tendon being wrapped by NFM or not.

It is envisaged by the invention that the NFM having core and shell made by electrospinning a coaxial double needle with different components added to the core and the shell can cause a quick release rate in the shell and a slow release rate in the core. The shell 30 having nano silver can inhibit the growth of microorganisms. The core 20 having HA can render the NFM non-sticky. As a result, both the properties of inhibiting the growth of microorganisms and being non-sticky are obtained by the NFM 10.

While the invention has been described in terms of preferred embodiments, those skilled in the art will recognize that the invention can be practiced with modifications within the spirit and scope of the appended claims.

What is claimed is:

1. A process of producing a nanofibrous membrane (NFM) having a core and a shell comprising the steps of:
(a) pouring dimethylformamide (DMF) and dichloromethane into a first flask;
(b) pouring about 1 to about 40 wt % of polycaprolactone (PCL) into the first flask to prepare a PCL solution therein;
(c) keeping the first flask in a predetermined temperature wherein volumetric ratio of DMF and dichloromethane is 1:9 to 9:1;
(d) pouring hyaluronic acid (HA) into a second flask to prepare an HA solution having 1.75 wt % of HA; and
(e) activating an electrospinning technique to process the PCL solution in the first flask and the HA solution in the second flask, thereby producing an NFM having a core and a shell.

2. The process of claim 1, wherein step (b) comprises the sub-steps of:
(b-1) adding about 0.01 to about 10 wt % of silver nitrate to the first flask; and
(b-2) placing the first flask in an ultraviolet box to be radiated for 3 hours to form nano silver by subjecting to optical reduction.

3. The process of claim 2, wherein volumetric ratio of DMF and dichloromethane is 2:8; 0.8 g of PCL is added to the first flask, and the PCL solution has 8 wt % of PCL and 0.5 wt % of silver nitrate.

4. The process of claim 1, wherein the PCL is about 1.75 wt % and step (d) comprises the sub-steps of:
(d-1) pouring about 0.5 wt % of polyethylene oxide (PEO) and about 10 g of formic acid into the second flask;
(d-2) sealing the second flask; and
(d-3) agitating the second flask by means of a magnetic member until a polymeric based solution is prepared in the second flask.

5. The process of claim 1, wherein step (e) comprises the sub-steps of:
(e-1) using a coaxial double needle to pierce into the first and second flasks respectively;
(e-2) drawing the PCL solution in the first flask and the HA solution in the second flask into a syringe sequentially;
(e-3) providing an adapter on the syringe to connect to an end of a Teflon pipe;
(e-4) providing a first stainless needle having a bore of 16-25 at an outer needle of the double needle at the other end of the Teflon pipe;
(e-5) providing a second stainless needle having a bore of 18-27 at an inner needle of the double needle at the other end of the Teflon pipe;
(e-6) securing the inner and outer needles to two syringe pumps respectively with different flow rates being set in the syringe pumps wherein flow rate of the HA solution containing the core is about 0.1-20 mL/hr, flow rate of the PCL solution containing the shell is about 0.3-30 mL/hr, the coaxial double needle is electrically connected to a positive terminal of a high voltage output, the inner and outer needles are concentric, and liquid flows out of the inner and outer needles respectively;
(e-7) grounding a collector for collecting nanoscale material; and
(e-8) adjusting the syringe pumps so that flow rate through the inner needle is about 1.0 mL/hr which is equal to flow rate through the outer needle wherein output voltage is 20 kV, and distance between each stainless needle and the collector is about 15 cm.

6. The process of claim 5, wherein a sharp point of the inner needle has a bore of 23, a sharp point of the outer needle has a bore of 18, and the collector is a diaphragm of aluminum.

7. The process of claim 2, wherein conditions of the ultraviolet box are 254 nm, 115V, 60 Hz, 0.7 A, and 0.1 J/cm².

8. The process of claim 1, wherein the PCL has a molecular weight of 1,000-1,000,000 Da.

9. The process of claim 1, wherein the NFM has a thickness of 1-5,000 nm.

10. The process of claim 1, wherein the NFM has a diameter of 10-300,000 nm.

11. The process of claim 1, wherein the NFM has a diameter of 10-300,000 nm.

12. The process of claim 1, wherein the NFM has a pore diameter of 50-50,000 nm.

13. A nanofibrous membrane (NFM) having a core and a shell produced by the process of claim 1, comprising:
a polycaprolactone (PCL) shell; and
a hyaluronic acid (HA) core surrounded by the PCL shell.

14. The nanofibrous membrane of claim 13, wherein the PCL shell contains silver nitrate.

15. The nanofibrous membrane of claim 13, wherein the HA core contains polyethylene oxide (PEO).

16. The nanofibrous membrane of claim 13, wherein the NFM has a diameter of 10-300,000 nm.

17. The nanofibrous membrane of claim 13, wherein the NFM has a pore diameter of 50-50,000 nm.

18. The nanofibrous membrane of claim 13, wherein the NFM has a porosity of 30-99%.
